(19) United States
(12) Patent Application Publication
(10) Pub. No.: US 2012/0095059 A1
(43) Pub. Date: Apr. 19, 2012

(54) Compositions and Methods for Treating Type II Diabetes and Related Disorders

(76) Inventors: Jorge Luis Rosado, Queretaro (MX); Miguel Angel Duarte-Vázquez, Queretaro (MX)

(21) Appl. No.: 13/086,213

(22) Filed: Apr. 13, 2011

Related U.S. Application Data
(60) Provisional application No. 61/323,788, filed on Apr. 13, 2010.

(51) Int. Cl. A61K 31/4439; A61P 3/10

(52) U.S. Cl. 514/342

(57) ABSTRACT

The present invention relates to methods and compositions for treating type II diabetes. This invention discloses an orally administrable pharmaceutical formulation, as convenient therapy to reduce glucose levels in blood, preventing the development of diabetes and an earlier exhausting of the pancreas. In certain embodiments, the invention provides orally administerable combination pharmaceutical formulations, comprising at least one insulin sensitizer (e.g., a glitazone such as pioglitazone or rosiglitazone), at least one sulfonylurea (e.g., glimepiride), and at least one biguanide (e.g., metformin or a pharmaceutically acceptable salt or ester thereof, such as metformin HCl). The present invention also provides methods of treating type II diabetes and related disorders in a mammal, suitably a human, by administering to a mammal in need thereof one or more of the compositions of the present invention.

### BACKGROUND OF THE INVENTION

1. Field of the Invention

The present invention is in the fields of medicine, pharmacy, and pharmaceutical formulations. More specifically, the invention is directed to methods and compositions for treating glycemic imbalance disorders, such as Type II diabetes and related disorders with a combination therapeutic, such as a composition comprising an insulin sensitizer (pioglitazone and/or rosiglitazone), glimepiride, and metformin.

2. Background

Type 2 diabetes is the most common form of diabetes, accounting for 90-95% of diagnosed patients. See International Diabetes Federation. Diabetes, Atlas. (3^{rd} Ed., 2007). The disease is characterized by impaired insulin secretion, insulin resistance in the liver, adipose tissue and skeletal muscle, and elevated glucose levels in blood (hyperglycemia). The degree and duration of hyperglycemia is a key determinant in the development of long-term complications of diabetes such as neuropathy, retinopathy, nephropathy, and cardiovascular events. These complications represent a major threat to public health. Therefore, adequate therapy is needed to improve glucose homeostasis and thus reduce the long-term micro- and macro-vascular complications of diabetes.

Type 2 diabetes is a chronic, progressive disease associated with high morbidity and mortality. The pathophysiology of type 2 diabetes mellitus is characterized by peripheral insulin resistance, impaired regulation of hepatic glucose production, and declining β-cell function in the pancreas. These events eventually lead to β-cell failure and a reduction in insulin secretion.

The primary events in the onset of diabetes are an initial deficit in insulin secretion and, in many patients, relative insulin deficiency in association with peripheral insulin resistance in the liver, adipose tissue, and skeletal muscle. These abnormalities contribute to abnormal glucose metabolism and loss of glucose homeostasis. The degree and duration of hyperglycemia is a key determinant in the development of long-term complications of diabetes.

Diet and exercise form the basis of all initial interventions programs for prevention and treatment of patients with type 2 diabetes. In patients with mildly elevated levels of fasting plasma glucose, type 2 diabetes is first treated by increasing physical activity, decreasing carbohydrate intake, and losing weight. It is sometimes possible to achieve long-term, satisfactory glucose control by following these guidelines. These interventions are the foundation of most metabolic therapies for the early stages of type 2 diabetes. However, when these tools are unable to achieve adequate glycemic control, pharmacological intervention is required.

If blood glucose levels remain elevated despite dietary control and the introduction of exercise, treatment with oral medication is usually recommended. Current therapeutic agents available for type 2 diabetes mellitus include sulfonylureas and related compounds, biguanides, thiazolidinediones, α-glucosidase inhibitors, and insulin.

Sulfonylureas were the first oral antidiabetic agents and they continue to be widely used worldwide. They stimulate insulin secretion by blocking ATP-sensitive potassium channels in the pancreatic β-cells. As a result, calcium channels open, thus increasing levels of cytoplastic calcium and stimulating insulin release. See Ligtenberg et al., Diabetes, Obesity and Metabolism 6:104-113 (1995). Sulfonylureas can produce an adequate glycemic control in recently diagnosed patients. However, normal glycemic control usually cannot be maintained over the long-term using sulfonylureas alone. Furthermore, prolonged insulin secretion mediated by sulfonylureas is associated with increased risk of cardiovascular disease.

Metformin (a biguanide) is one of the most widely used oral antidiabetic agents for the treatment of type 2 diabetes. Metformin exerts its glucose-lowering effect by decreasing hepatic glucose output and improving insulin sensitivity. Metformin does not affect insulin secretion. Its effects on glycemic control are achieved mainly by reducing fasting plasma glucose. It does not cause hypoglycemia, nor does not stimulate insulin secretion by the pancreas. See DeFronzo et al., N Engl J Med 333: 541-595 (1995).

The use of metformin alone (as monotherapy) is usually inadequate to maintain glycemic control in some patients, thus they require the addition of another therapy to maintain glycemic control. Current treatment guidelines recommend the addition of sulfonylureas in such patients. However, the addition of sulfonylureas further increases hyperinsulinemia (increased levels of insulin in the plasma due to increased secretion of insulin by the pancreas) and improvements in glycemic control as a result of adding a sulfonylurea are usually not sustained long-term. See Wright et al., Diabetes Care 25: 330-336 (2002). Inzucchi et al., JAMA 287:360-372 (2002).

α-Glucosidase inhibitors delay the absorption of carbohydrate by the intestine by inhibiting the α-glucosidase enzymes at the ciliated border of the intestine, thereby minimizing the postprandial rise in blood glucose. When used alone in monotherapy, α-glucosidase inhibitors do not cause hypoglycemia. This class of drugs includes three compounds: acarbose, miglitol and voglibose. Acarbose is the most prescribed α-glucosidase inhibitor. Acarbose can be used alone or in combination with other antidiabetic oral drug such as metformin and thiazolidinediones. See Willms and Ruge, Diabetic Medicine 16: 755-761.

Thiazolidinediones promote glucose utilization by activation of PPARγ receptors, mainly in adipose tissue. They may also inhibit hepatic glucose production. The thiazolidinediones sensitize and enhance the effect of insulin in skeletal muscle, adipose and hepatic tissues, without increasing pancreatic secretion of insulin. They also reduce circulating concentrations of pro-inflammatory cytokines that promote insulin resistance. See Stumvoll et al., Lancet 365:1533-1346 (2005). In addition to reducing insulin resistance, thiazolidinediones also improve beta-cell dysfunction, thereby addressing two principal underlying metabolic abnormalities in most patients with type 2 diabetes mellitus. See Matthews et al., Diabetologia 42:A228 (1999). The major concern with this class of drugs is that they cause hepatotoxicity, especially troglitazone. Only pioglitazone does not share this hepatotoxic profile.

The United Kingdom Prospective Diabetes Study (UKPDS) found that type 2 diabetes is a progressive disease with slowly declining β-cell function, leading to decreasing insulin secretion over time. See Rudensky et al., Diabet. Med. 5:36-41 (1988). Therapy with any single oral antidiabetic agent (monotherapy) becomes less effective over time, and the addition of another antidiabetic agent eventually becomes necessary to maintain glycemic control.

Most available antidiabetic agents have been used in combination to treat type 2 diabetes. A rational choice for combination therapy is the combination of an agent that increases insulin with one that enhances sensitivity to insulin and lowers glucose production. This combination of agents can correct most of the pathophysiological defects found in type 2 diabetic patients more effectively than the use of a single agent. See Blonde et al., Diabetes, Obesity and Metabolism, 4: 368-375 (2002); Horton et al., Diabetes Care, 23: 1660-1665 (2000); and Jones et al., Diabetes, Obesity and Metabolism, 5: 163-170 (2003).

The appropriate choice of antidiabetic agents for combination treatment of type 2 diabetes depends upon both the pharmacological properties of the medication and the clinical characteristics of the patient. See Chehade and Mooradian, Drugs 60:95-113 (2000). The most commonly used combination therapy is metformin plus a sulfonylurea. See De Fronzo, Ann. Intern. Med., 131:281-303 (1999). These agents have complementary mechanisms of action: sulfonylureas enhance insulin secretion, whereas metformin increases peripheral and hepatic insulin sensitivity. Several studies have shown that this combination provides greater glycemic control than is obtained with either agent alone.

Another combination therapy that is extensively used is metformin plus thiazolidinediones (or glitazones). Glitazones exert their anti-hyperglycemic effect by reducing insulin resistance, and sensitizing liver, muscle and adipose tissue to the action of insulin. The modes of action of glitazones and metformin are different, but complementary. Metformin reduces hepatic glucose production and increases peripheral and hepatic insulin sensitivity. See Fonseca et al., JAMA, 283: 1695-1702 (2000); Jones et al., Diabetes, Obesity and Metabolism 5: 163-170 (2003); Saltiel and Olefsky, Diabetes, 45: 1661-1669 (1996).

After treatment with the above described substances alone or in combination, some patients with type 2 diabetes still have HbA1c (glycated hemoglobin) levels greater than 7%, indicating a failure to reach the goals of treatment. Moreover, patients having HbA1c levels greater than 7% have a high risk for cardiovascular disease and other complications of diabetes. Therefore, the addition of a third agent is required to achieve adequate glucose control in such patients with advanced type 2 diabetes. See Triplitt et al., Diabetes Care, 29: 2371-2377 (2006).

3. Related Art

Blonde et al., Diabetes, Obesity and Metabolism, 4:368-375 (2002) compared the efficacy, safety and tolerability of a fixed combination of glyburide (glibenclamide, a sulfonylurea) plus metformin with those of glyburide or metformin alone, in patients with type 2 diabetes inadequately controlled by sulfonylurea alone, diet and exercise. After 16 weeks of treatment, the use of glyburide plus metformin produced significantly better glycemic control than monotherapy with either agent alone.

In a randomized, double-blind, double-dummy study, Charpentier et al.,

Diabetic Medicine, 18:828-834 (2001) compared the effect of glimepiride in combination with metformin with monotherapy of each drug in type 2 diabetic patients. Combination therapy was significantly more efficient in controlling HbA1c levels, fasting blood glucose, and postprandial blood glucose, than either glimepiride or metformin alone. However, the incidence of symptomatic hypoglycemia was higher in the combination group than in either monotherapy group.

Jones et al., Diabetes, Obesity and Metabolism, 5:163-170 (2003) evaluated the efficacy of the insulin sensitizer rosiglitazone and near maximal doses of metformin (2.5 g/day) in obese type 2 diabetes mellitus patients. The authors found that the addition of rosiglitazone to a metformin regimen improves glycemic control, insulin sensitivity, and beta-cell function in obese type 2 diabetic patients whose blood sugar is inadequately controlled with metformin alone.

De Rosa et al., Diabetes Research and Clinical Practice, 69:5-13 (2005) compared the effect of a combination of pioglitazone and glimepiride, or rosiglitazone and glimepiride, in diabetic patients with the metabolic syndrome. They enrolled 91 diabetic patients in a randomized, controlled, double-blind clinical study. All patients took a fixed dose of glimepiride (4 mg/d). Patients were randomized to receive pioglitazone (15 mg/d) or rosiglitazone (4 mg/d) for 12 months. After this period, a significant decrease in HbA1c, mean fasting plasma glucose, and posprandial plasma glucose was observed in both treatment groups. Therefore, the addition of rosiglitazone or pioglitazone to a glimepiride regimen improved glycemic control compared to glimepiride monotherapy.

Rosenstock et al., Diabetes Care, 25:1529-1533 (2002) compared the efficacy of monotherapy in patients with type 2 diabetes mellitus using nateglinide alone, hoglitazone alone, and a combination of nateglinide and troglitazone. Nateglinide or troglitazone alone were effective in decreasing HbA1c levels. The reduction in HbA1c was of 0.6% and 0.8% in the nateglinide and troglitazone treatment groups, respectively. The reduction in both groups was not adequate to achieve HbA1c levels of less than 7%. In contrast, the combination of nateglinide and troglitazone reduced HbA1c levels to less than 7% in 66% of patients receiving the combination therapy.

WO 98/57634 discloses a method for treating diabetes employing a combination of thiazolidinedione and metformin. The thiazolidinedione is selected from the group comprising troglitazone, ciglitazones, pioglitazone, or englitazone, and may be employed in daily dosages of 2 to 12 mg per day. Metformin may be employed at daily dosages ranging from 500 to 3000 mg per day.

WO 97/17975 discloses a combination of glibenclamide and metformin in a 1:100 ratio, with a daily dosage of 15 mg glibenclamide and 1500 mg metformin. This specification discloses that this combination is used to treat the most severe cases of diabetes, particularly in patients who have been unsuccessfully treated with a combination of glibenclamide-metformin HCl in weight ratio higher than 1:100.

U.S. Pat. Ser. No. 09/353,141 discloses formulations containing metformin and glyburide wherein the glyburide is of a particular particle size.

EP 0749751A2 discloses a combination of a thiazolidinedione insulin sensitivity enhancer (such as pioglitazone) and metformin.

U.S. Pat. No. 5,922,769 discloses the use of a combination consisting of glibenclamide and metformin in a specific ratio as medicament for the treatment of diabetes mellitus type 2. The medication is in the form of tablet containing 5 mg of glibenclamide and 500 mg of metformin. The treatment is recommended for patients who have been unsuccessfully treated with a combination of agents, not in naïve patients or patients who were unsuccessfully treated with monotherapy.

U.S. Pat. No. 6,586,438 B2 discloses a low dose pharmaceutical formulation for treating type 2 diabetes in drug naïve patients, which includes metformin (preferably employed in reduced amounts compared to that employed in generally accepted medical practice) and another antidiabetic agent, such as a sulfonylurea. The formulation has substantially equivalent efficacy in treating type 2 diabetes compared to prior antidiabetic formulations containing metformin, but with substantially reduced side effects. These formulations have the disadvantage that they are directed mainly to naive patients, which represents a small proportion of total diabetic type II population.

U.S. Pat. No. 5,859,037 discloses a combination of a sulfonylurea antidiabetic agent and a glitazone antidiabetic agent useful for treating diabetes mellitus and improving glycemic control. The sulfonylurea is selected from glisoxepid, glyburide, acetohexamide, chlorpropamide, glibornuride, tolbutamide, tolazamide, glipizide, gliclazide, gliquidone, glyhexamide, phenbutamide, and tolcyclamide. The glitazone is selected from troglitazone, ceglitazone, pioglitazone, anglitazone, TA 174, and BRL 49653. More specifically, this invention is related to the use of troglitazone and glyburide. The combination claimed in this patent has some disadvantages, one of which is that it contains troglitazone, which was withdrawn from the U.S. market. Also, the sulfonylureas included in this invention have the disadvantage of causing severe episodes of hypoglycemia.

U.S. Pat. No. 6,011,049, which is a continuation-in-part of the above cited U.S. Pat. No. 5,859,037, discloses a combination of a glitazone antidiabetic agent, a biguanide antidiabetic agent, and a sulfonylurea antidiabetic agent, useful for treating diabetes mellitus and improving glycemic control.

Therefore, there remains a need in the art for compositions and methods for treating type II diabetes and related disorders.

BRIEF SUMMARY OF THE INVENTION

The present invention provides methods of treating or preventing glycemic imbalance disorders in a mammal, including, but not limited to, type II diabetes mellitus, type I diabetes, diabetes insipidus, hyperglycemia, hypothyroidism, and the like.

Two options are recommended for patients who fail to achieve adequate glucose control with the oral combinations described above: 1) the addition of a third oral agent, i.e. thiazolidinediones, or 2) the addition of long acting insulin. The use of insulin therapy has difficulties such as concerns of hypoglycemia, administering a complex and traumatic regimen of insulin injections, and erratic absorption of insulin. Therefore, the addition of a third oral agent is the best therapeutic approach.

Metformin has been shown to lower blood glucose levels by sensitizing the liver to the effects of insulin, thus suppressing hepatic glucose output. Thiazolidinediones, like pioglitazone, improve insulin sensitivity by enhancing insulin-mediated glucose disposal. Glimepiride stimulates β-cells, thus enhancing insulin secretion. Sulfonylureas (such as glimepiride), biguanides (such as metformin) and glitazones (such as pioglitazone) have different, but complementary, mechanisms of action. A combination therapy comprising these drugs will have a synergistic effect on the reduction of blood glucose levels and HbA1c production, thereby diminishing the progression and occurrence of diabetes type 2 complications.

In one aspect, the present invention is directed to an oral formulation for the treatment of diabetes and related disorders comprising an insulin sensitizer, glimepiride, and sustained-release metformin.

In some embodiments, the oral formulation a solid formulation, which may be selected from the group consisting of capsules, tablets, pills, powders, particles, and granules.

In some embodiments, the oral Formulation is a liquid formulation, which may be selected from the group consisting of emulsions, solutions, suspensions, syrups, and elixers.

In some embodiments, the oral formulation comprises from about 0.5 mg to about 40 mg of glimepiride.

In some embodiments, the oral formulation comprises from about 2 mg to about 120 mg of insulin sensitizer, such as a glitazone including, but not limited to, pioglitazone and rosiglitazone.

In some embodiments, the oral formulation comprises from about 500 mg to about 5000 mg of metformin (or a pharmaceutically acceptable salt thereof, such as metformin hydrochloride), which can optionally be in a sustained-release or extended-release formulation.

The present invention is also directed to a kit comprising at least one oral formulation of the present invention and information containing instructions to administer the oral formulation once per day, twice per day, three times per day, or four times per day to a subject in need thereof.

The present invention is also directed to methods for treating diabetes and related disorders in a mammal, such as a human, comprising administering to said mammal an oral formulation comprising an insulin sensitizer, glimepiride, and metformin, or an oral formulation of the present invention.

In some embodiments, the method comprises administering a daily dose of glimepiride from about 0.5 mg to about 40 mg.

In some embodiments, the method comprises administering a daily dose of the insulin sensitizer from about 2 mg to about 120 mg.

In some embodiments, the method comprises administering a daily dose of metformin (or a pharmaceutically acceptable salt thereof, such as metformin hydrochloride) from about 500 mg to about 5000 mg, which may optionally be in a sustained-release or extended-release formula.

In some embodiments, the insulin sensitizer is a glitazone, such as pioglitazone or rosiglitazone.

Thus, in one aspect the invention generally provides pharmaceutical compositions suitable for oral administration for the treatment of diabetes and related disorders, comprising at least one insulin sensitizer, glimepiride, and sustained-release metformin or pharmaceutically active salt or ester thereof. In certain such embodiments, the oral formulation is a solid formulation which may take the form of capsules, tablets, pills, powders, particles, granules and the like. In related such embodiments, the oral formulation may take the form of a liquid formulation such as an emulsion, a solution, a suspension, a syrup, an elixer and the like. In suitable embodiments of this aspect of the invention, the oral formulation comprises from about 0.5 mg to about 40 mg of glimepiride; from about 2 mg to about 120 mg of the insulin sensitizer (which suitably is a glitazone, such as pioglitazone or rosiglitazone), and from about 500 mg to about 5000 mg of metformin hydrochloride.

In related aspects, the invention provides kits for use in treating Type II diabetes, comprising the oral formulations of the present invention and a label containing instructions to administer the oral formulation once per day, twice per day, three times per day, or four times per day to a subject in need thereof

In another aspect, the invention provides methods for treating Type II diabetes and related disorders in a mammal (suitably, a human), comprising administering to the mammal an oral formulation, such as an oral formulation of the present invention, comprising at least one insulin sensitizer, glimepiride, and sustained-release metformin or pharmaceutically active salt or ester thereof Suitable such methods comprise, for example, administering to the mammal a daily dose of glimepiride of about 0.5 mg to about 40 mg (suitably about 2 mg), a daily dose of the insulin sensitizer (which suitably is a glitazone, such as pioglitazone or rosiglitazone) of about 2 mg to about 120 mg (suitably about 15 mg), and a daily dose of metformin, or pharmaceutically active salt or ester thereof (e.g., metformin HCl) of about 500 mg to about 5000 mg (suitably about 500 mg to about 850 mg, and more suitably about 500 mg, about 600 mg, about 650 mg, about 700 mg, about 700 mg, about 750 mg, about 800 mg, or about 850 mg).

Other features and advantages of the invention will be apparent from the following detailed description, and from the examples and claims. The disclosed materials, methods, and examples are for illustrative purposes only and are not intended to be limiting. Skilled artisans will appreciate that methods and materials similar or equivalent to those described herein can be used to practice the invention.

Unless otherwise defined, all technical and scientific terms used herein have the meaning commonly understood by one skilled in the art to which this invention belongs. All publications, patent applications, patents, and other references mentioned herein are incorporated by reference in their entirety. In case of conflict, the present specification, including definitions, will control.

### BRIEF DESCRIPTION OF THE DRAWINGS

FIG. 1 provides the change from baseline in HbA1c after 12 weeks treatment with glimepiride alone (Glime), pioglitazone alone (Pio), pioglitazone and glimepiride in combination (Pio-Glime), or the expected change from baseline HbA12c after 12 weeks of treatment with pioglitazone, glimepiride, and metformin (Pio-Glime-Met) in combination. The bar corresponding to pioglitazone-glimepiride-metformin represents the predicted change upon treatment with the combination pharmaceutical formulation of the present invention.

FIG. 2 provides the mean decrease from baseline in FBG (mg/dL) after 12 weeks treatment with glimepiride alone (Glime), pioglitazone alone (Pio), pioglitazone and glimepiride in combination (Pio-Glime), or the expected mean decrease in baseline FBG (mg/dL) after 12 weeks treatment with pioglitazone, glimepiride, and metformin (Pio-Glime-Met) in combination. The bar corresponding to pioglitazone-glimepiride-metformin represents the predicted change upon treatment with the combination pharmaceutical formulation of the present invention.

### DETAILED DESCRIPTION OF THE INVENTION

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods and materials are described hereinafter.

### Definitions

About: As used herein when referring to any numerical value, the term “about” means a value of ±10% of the stated value (e.g., “about 50° C.” encompasses a range of temperatures from 45° C. to 55° C., inclusive; similarly, “about 100 mM” encompasses a range of concentrations from 90 mM to 110 mM, inclusive).

Disease, disorder, condition: As used herein, the terms “disease” or “disorder” refer to any adverse condition of a human or animal including tumors, cancer, allergies, addiction, autoimmunity, infection, poisoning or impairment of optimal mental or bodily function. “Conditions” as used herein includes diseases and disorders but also refers to physiologic states. For example, fertility is a physiologic state but not a disease or disorder; hence, compositions suitable for preventing pregnancy by decreasing fertility would therefore be described herein as a treatment of a condition (fertility), but not a treatment of a disorder or disease. Other conditions encompassed by the use of that term herein will be understood by those of ordinary skill in the art.

One, a, or an: When the terms “one,” “a,” or “an” are used in this disclosure, they mean “at least one” or “one or more,” unless otherwise indicated. As such, the terms “a” (or “an”), “one or more,” and “at least one” can be used interchangeably herein.

Treatment: As used herein, the terms “treatment,” “treat,” “treated” or “treating” refer to prophylaxis and/or therapy, particularly wherein the object is to prevent or slow down (lessen) an undesired physiological change or disorder, such as the development and/or progression of cardiovascular disease. Beneficial or desired clinical results include, but are not limited to, alleviation of symptoms, diminishment of the extent of disease, stabilized (i.e., not worsening) state of disease, delay or slowing of disease progression, amelioration or palliation of the disease state, and remission (whether partial or total), whether detectable or undetectable. “Treatment” can also mean prolonging survival and/or increased quality of life as compared to expected survival and/or quality of life if not receiving treatment. Those in need of treatment include those already with the condition or disorder (e.g., cardiovascular disease) as well as those prone to have the condition or disorder or those in which the condition or disorder is to be prevented. By “subject” or “individual” or “animal” or “patient” or “mammal,” is meant any subject, particularly a mammalian subject, for whom diagnosis, prognosis, or therapy is desired. Mammalian subjects include humans and other primates, domestic animals, farm animals, and zoo, sports, or pet animals such as dogs, cats, guinea pigs, rabbits, rats, mice, horses, donkeys, mules, burros, cattle, cows, and the like.

### Overview

Type 2 diabetes has a complex pathophysiology, combining a defect in insulin secretion by the pancreas, increased glucose production by the liver, and reduced insulin-mediated glucose uptake by the skeletal muscle. Each of these abnormalities can be partially reversed by a specific drug, an agent promoting insulin secretion (e.g., a sulfonylurea), an agent blocking hepatic glucose production (e.g., metformin, or a pharmaceutically acceptable salt thereof, e.g., metformin hydrochloride), and an insulin sensitizer thiazolidinedione (such as a glitazone, e.g., pioglitazone and/or rosiglitazone).

Thus, in certain embodiments, the present invention provides a triple oral therapy useful for the treatment of diabetes and related disorders, incorporating (a) a therapeutically effective amount of a sulfonylurea, and more specifically glimepiride, which does not produce hypoglycemia; (b) a therapeutically effective amount of metformin or a pharmaceutically acceptable salt thereof, e.g., metformin hydrochloride; and (c) a therapeutically effective amount of an insulin sensitizer, such as a glitazone, e.g., pioglitazone or rosiglitazone.

Glimepiride as used in the formulation of the invention is a direct insulin secretagogue. Indirectly, it also increases insulin secretion in response to glucose. Treatment with glimepiride is associated with a lower incidence of hypoglycemia than glibenclamide.

Metformin acts via a mechanism which is distinct from sulfonylureas.

Metformin decreases hepatic glucose output by altering the intracellular energy metabolism and stimulating peripheral glucose uptake. Metformin also improves blood glucose control without increasing the risk of hypoglycemia.

Insulin sensitizers, such as pioglitazone or rosiglitazone, increase peripheral glucose utilization by binding and activating nuclear peroxisomal proliferator-activated receptors-gamma (PPAR-γ).

Not wishing to be bound by any particular theory, it is believed that insulin sensitizers (such as pioglitazone and/or rosiglitazone), glimepiride, and metformin have different but complimentary actions affecting glycemic control. Glimepiride stimulates insulin secretion. Thus, the inclusion of an insulin sensitizer, such as pioglitazone, will optimize insulin utilization. Therefore, less insulin is required to be manufactured by the beta cells of the pancreas. As a result, lower doses of sulfonylurea may be used, avoiding early exhausting of the pancreas and also producing less adverse effects. In the same way, the inclusion of the insulin sensitizer to the formulation will reduce the amount of metformin required to achieve adequate glycemic blood levels. Furthermore, adverse side effects, such as hypoglycemia and gastrointestinal effects, are reduced.

Certain of the pharmaceutical formulations described herein will increase insulin secretion by the action of glimepiride. The presence of metformin and pioglitazone will optimize the insulin action and glucose utilization by the body. Ire addition, triple therapy with certain of the formulations of the present invention, such as those comprising glimepiride/pioglitazone/metformin, will increase the glucose delivered to the blood which can be utilized by the body.

### Compositions

In some embodiments, the present invention provides pharmaceutical compositions useful for the treatment of symptoms of blood sugar imbalance associated with various diseases and disorders such as type II diabetes mellitus and related disorders, such as, for example, type I diabetes, diabetes insipidus, hyperglycemia, hypothyroidism, hypercholesterolemia, hypertriglyceridemia, cardiovascular events, and the like, in a mammal.

In some embodiments, the present invention provides pharmaceutical compositions comprising an insulin sensitizer, glimepiride, and metformin, which may optionally be in a sustained-release or extended release form. In some embodiments, the insulin sensitizer is a glitazone, suitably pioglitazone and/or rosiglitazone.

In some embodiments, the active compounds are combined in a single formulation, along with one or more pharmaceutically acceptable carriers, diluents, or excipients that will be readily familiar to those of ordinary skill in the relevant arts. The combination of the three agents into a single dosage form provides the additional advantages of using reduced dosages of the active ingredients, increasing patient compliance, and consequently, having a greater effect on glycemic control.

In some embodiments, the compositions of the invention may be formulated into forms for oral administration, including solid dosage forms or liquid dosage forms. In alternative embodiments, the compositions of the invention may be formulated into forms for direct administration to the mucosa, including the buccal mucosa (i.e., buccal administration) or oral mucosa under the tongue (i.e., sublingual administration). Solid dosage forms for oral administration include capsules, tablets, pills, powders, particles and granules. In such solid dosage forms, the compositions of the invention are mixed with at least one pharmaceutically acceptable excipient or carrier such as (a) fillers or extenders such as starches, lactose, sucrose, glucose, mannitol, dicalcium phosphate and microcrystalline cellulose; (b) binders such as sodium carboxymethylcellulose, alginates, gelatin, polyvinylpyrrolidone, and acacia; (c) disintegrating agents such as agar-agar, calcium carbonate, potato or tapioca starch, alginic acid, certain silicates, sodium carboxymethyl cellulose, pregelatinized starch and sodium starch glycolate; (d) lubricants such as calcium stearate, magnesium stearate, stearic acid, solid polyethylene glycols, sodium lauryl sulfate, and mixtures thereof; and/or (e) glidants such as talc, silicon dioxide and starch. In the case of capsules, tablets and pills, the dosage form may also comprise buffering agents. Solid compositions of a similar type may also be employed as fillers in soft and hard filled gelatin capsules using such excipients as lactose or milk sugar as well as high molecular weight polyethylene glycols, oils and the like. The solid dosage forms of tablets, diagees, capsules, pills, and granules can be prepared with coatings or shells such as enteric coatings and other coatings that are well known in the pharmaceutical formulating art. The solid dosage forms also may optionally contain opacifying, coloring and/or flavoring agents, and can also be formulated such that they release the active ingredient(s) only, or preferentially, it a certain part of the intestinal tract, optionally in a delayed manner (see U.S. Pat. No. 5,271,946, the disclosure of which is incorporated herein by reference in its entirety). Examples of embedding compositions which can be used include polymeric substances and waxes. The active compounds can also be in micro-encapsulated form, if appropriate, with one or more of the above-mentioned excipients.

In certain such embodiments, a single dose of the oral formulation comprises glimepiride in an amount that is pharmaceutically effective, e.g., about 0.5 mg to about 40 mg, about 1 mg to about 10 mg, or about 2 mg to about 6 mg. In suitable such embodiments, a single dose of the oral formulation comprises about 2 mg glimepiride.

In certain such embodiments, the oral formulation comprises an insulin sensitizer, e.g., a glitazone such as pioglitazone, in an amount that is pharmaceutically effective, e.g., about 2 mg to about 120 mg, about 10 mg to about 80 mg, or about 15 mg to about 60 mg. In suitable such embodiments, a single dose of the oral formulation comprises about 15 mg insulin sensitizer.

In certain such embodiments, the oral formulation comprises metformin, suitably metformin HCl or any pharmaceutically acceptable salt or ester form of metformin, in an amount that is pharmaceutically effective, e.g., about 500 mg to about 5000 mg metformin, or about 500 mg to about 3000 mg metformin. In suitable such embodiments, a single dose of the oral formulation comprises about 500 mg or about 850 mg metformin. The metformin or salt/ester form thereof may be in an immediate-release formulation, or optionally may be in a sustained- or extended-release formulation such as those described herein.

Chewable and/or Orally Dissolving Formulations

### Chewable Formulations

In addition to the solid dosage forms disclosed throughout, the present invention also provides chewable oral formulations. In certain such embodiments, the formulations will comprise (or consist essentially of) a therapeutically effective amount of one or more of the compositions of the present invention, such as a combination formulation of one or more insulin sensitizers (e.g., one or more glitazones such as pioglitazone), one or more sulfonylureas (e.g., glimepiride) and one or more biguanides (e.g., metformin, suitably metformin HCl), along with suitable excipients that allow the formulations to be chewed by the patient. In one such embodiment, the chewable formulations comprise a formulation comprising a therapeutically effective amount of each of pioglitazone, glimepiride and metformin HCl. Additional active agents, such as those described herein, can also optionally be added to the chewable formulations. The amounts of one or more insulin sensitizers, one or more sulfonylureas and one or more biguanides, and other optional active agents, in the chewable formulations of the present invention are readily determinable by those of ordinary skill in the art, and include those amounts and combinations described herein. For example, certain exemplary chewable formulations of the present invention comprise (or consist essentially of) about 2-120 mg of an insulin sensitizer, about 0.5-40 mg of a sulfonylurea, and about 200-5000 mg of a biguanide, with optimal doses and amounts of each of these active ingredients capable of being determined by one of ordinary skill based on the guidance provided herein and knowledge that is available in the art, and routine methods of dosage optimization that will be familiar to the ordinarily skilled clinician and pharmacist. Such chewable formulations are especially useful in patient populations where compliance is an issue, such as children, the elderly, and patients who may have difficulty swallowing or using spray/inhalable formulations.

The formulations may also contain colorants to improve the appearance of the chewable formulations, especially since an attractive coloration imparted by a colorant may improve patient compliance. The relative amounts of the colorants selected will vary depending upon the particular hue of the individual colorants and the resultant color desired.

Any standard pharmaceutically acceptable excipient can be used in the chewable tablet formulations which provides adequate compression such as diluents (e.g., mannitol, xylitol, maltitol, lactitol, sorbitol, lactose, sucrose, and compressible sugars such as DiPac® (dextrinized sucrose), available from Austin Products Inc. (Holmdel, N.J.), binders, disintegrants, splitting or swelling agents (e.g., polyvinyl polypyrrolidone, croscarmellose sodium (e.g., Ac-Di-Sol available from FMC BioPolymer, Philadelphia, Pa.), starches and derivatives, cellulose and derivatives, microcrystalline celluloses, such as Avicel™ PH 101 or Avicel™ CE-15 (a microcrystalline modified with guar gum), both available from FMC BioPolymer, (Philadelphia, Pa.), lubricating agents (e.g., magnesium stearate), and flow agents (e.g., colloidal silicon dioxide, such as Cab-O-Sil M5® available from Cabot Corporation, Kokomo, Ind.).

Suitable amounts of sweetener (e.g., sucralose, aspartame, polysaccharides, xylitol, sorbitol, and other sweeteners that will be readily familiar to those of ordinary skill) used in the chewable formulations, will be familiar to, and can be readily determined by, those skilled in the art. In certain embodiments, the sweetener is present in an amount from about 0.05% to about 5.0% (e.g., about 0.05%, about 0.1%, about 0.125%, about 0.15%, about 0.2%, about 0.3%, about 0.4%, about 0.5%, about 0.6%, about 0.7%, about 0.8%, about 0.9%, about 1%, about 1.25% about 1.5%, about 1.75%, about 2%, about 2.25%, about 2.5%, about 2.75%, about 3%, about 3.25%, about 3.5%, about 3.75%, about 4%, about 4.25%, about 4.5%, about 4.75% or about 5%). Those or ordinary skill in the art will appreciate that the amount of sweetener may vary depending on the strength of the particular sweetener used and the levels approved by the regulatory authorities for use in pharmaceutical products.

Suitable cyclodextrins for use in the chewable formulations of the present invention include α, β, or γ cyclodextrins, or an alkylated or hydroxyalkylated derivatives thereof, such as heptakis (2,6-di-o-methyl)-β-cyclodextrin (DIMEB), randomly methylated β-cyclodextrin (RAMEB), and hydroxypropyl β-cyclodextrin (HPβCD). A suitable cyclodextrin is β-cyclodextrin (available from Cerestar USA, Inc., Hammond, Ind. or from Roquette America, Inc., Keokuk. Iowa under the trade name Kleptose™). If desired, the complex of the active substance(s) with cyclodextrin can be prepared in advance, for example, by malaxating or granulating the insulin sensitizer, sulfonylurea and/or biguanide, and any additional active substance(s), with the cyclodextrin in the presence of water, or by preparing an aqueous solution containing the insulin sensitizer, sulfonylurea and/or biguanide and any additional active substance(s) and the cyclodextrin in the desired molar ratio. Alternatively, the insulin sensitizer, sulfonylurea and/or biguanide and any additional active substance(s) and the cyclodextrin can be simply mixed with other excipients and adjuvants. The molar ratio of the insulin sensitizer, sulfonylurea and/or biguanide and any additional active substance(s) to cyclodextrin is suitably from about 1.0:1.0 to about 4.0:1.0.

A typical manufacturing process for making either a single layer or bi-layer chewable tablet generally involves blending of the desired ingredients to form a uniform distribution of the insulin sensitizer, sulfonylurea and/or biguanide (and any other active agent(s)), excipients (e.g., colorants and flavoring agents as well as others). If desired, an inclusion complex of the insulin sensitizer, sulfonylurea and/or biguauide and any other active agent(s) and cyclodextrin (e.g., β-cyclodextrin) may be formed prior to blending into the mixture by malaxating the insulin sensitizer, sulfonylurea and/or biguanide and any other active agent(s) and cyclodextrin in the presence of water in a planetary mixer for about 20 minutes. The mixture is then dried in a drying oven. After drying, the complex is mixed with any color/flavoring blend. The blend is then compressed into a single layer or bi-layer tablet using standard methods well-known to those skilled in the art (e.g., Kilian T-100 tablet press or Courtoy 292/43 rotary bi-layer press). The colorants and flavoring agents may be added to both layers to form a uniform presentation of the tablet. Methods for preparation of chewable tablets and various components for use in the tablets can be found throughout the detailed description section and the Examples of U.S. Patent Publication No. 2003/0215503, the disclosure of which is incorporated by reference herein for all purposes. Additional chewable/orally dissolving tablets, and methods for their manufacture, are disclosed in U.S. Patent Publication No. 2004/0265372 and U.S. Pat. No. 6,270,790, the disclosures of each of which are incorporated by reference herein for all purposes.

### Orally Disintegrating Tablets

In another embodiment, the present invention provides orally disintegrating/orodispersible tablets, such as those disclosed in U.S. Pat. No. 6,723,348, the disclosure of which is incorporated herein by reference in its entirety for all purposes. The orally disintegrating/orodispersible tablets suitably disintegrate in the buccal cavity upon contact with saliva forming an easy-to-swallow suspension. Such tablets comprise (or consist essentially of) one or more compositions of the invention, suitably one or more insulin sensitizers, sulfonylureas and/or biguanides such as those described herein, and optionally, one or more additional active agents (such as those described herein), in the form of coated granules, and a mixture of excipients comprising at least one disintegrating agent, a soluble diluent agent, a lubricant and optionally a swelling agent, an antistatic (fluid flow) agent, a permeabilising agent, taste-masking agents/sweeteners, flavoring agents and colors. The amounts of insulin sensitizer, sulfonylurea and/or biguanide, other optional active agents (e.g., insulin), and sweetening agents in the orally disintegrating tablet formulations of the present invention are readily determinable by those of ordinary skill in the art, and include those amounts and combinations described herein.

In suitable embodiments, the particles/granules of insulin sensitizer, sulfonylurea and/or biguanide (and any other optional active agents) have a particle size such that about 100% of the particles have an average size of less than about 50 μm. In suitable such embodiments, the insulin sensitizer, sulfonylurea and/or biguanide (and any other optional active agents) are present as coated granules.

In one embodiment, the disintegrating tablets according to the invention comprise coated granules of pioglitazone, glimepiride and metformin HCl (and optionally, one or more additional active agents), and a mixture of excipients, the ratio of the mixture of excipients to the coated granules suitably being about 0.4:1 to about 9:1, more suitable about 1.5:1 to about 5:1, or about 2 to 3 parts by weight, the mixture of excipients suitably comprising: at least one disintegrating agent, a soluble diluent agent, a lubricant, and optionally a permeabilising agent, a swelling agent, an antistatic agent, flavoring agents and one or more coloring agents.

In suitable such embodiments, the disintegrating agent is selected from the group consisting of croscarmellose, available as e.g. Ac-di-sol™, crospovidone available as e.g. Kollidon CL™, sodium starch glycolate and mixtures thereof.

According to one embodiment of the invention, the soluble diluent is a polyol having less than 13 carbon atoms and being either in the form of a directly compressible product with an average particle size of about 100 to 500 μm, or in the form of a powder with an average particle size of less than about 100 μm, this polyol suitably being selected from the group consisting of mannitol, xylitol, sorbitol and maltitol. The proportion of disintegrating agent suitably is from about 3 to about 15% by weight, e.g., about 5 to about 15% by weight, and in the case of a mixture, each disintegrating agent being present between about 1 and about 10% by weight, e.g., about 5 to about 10% by weight, and the proportion of soluble diluent agent being about 30 to about 90% by weight, e.g., about 40 to about 60% by weight, based in each case on the weight of the tablet.

Suitable lubricants for use in the disintegrating tablets include, but are not limited to, magnesium stearate, stearic acid, sodium stearyl fumarate, micronised polyoxyethyleneglycol (micronised Macrogol 6000), leukine, sodium benzoate and mixtures thereof. The amount of lubricant generally is from about 0 to about 3%, e.g., from about 1 to about 2% by weight, based on the weight of the tablet. The lubricant can be dispersed within the mixture of excipients, or according to one embodiment, sprayed over the outer surface of the tablet. Thus, according to one embodiment of the disintegrating tablets of the invention, the lubricant is in powder form and is, at least in part, disposed on the surface of the tablets.

The permeabilising agent allows the creation of a hydrophilic network which facilitates the penetration of saliva and hence assists the disintegration of the tablet. Suitable permeabilising agent include, but are not limited to, silica with a high affinity for aqueous solvents, such as colloidal silica (Aerosil™), precipitated silica (Syloid™ FP 244), maltodextrins, β-cyclodextrins and mixtures thereof. The amount of permeabilising agent suitably is between about 0 and about 5%, e.g., from about 0.5 to about 2% by weight, based on the weight of the tablet.

A swelling agent can be incorporated in the mixture of excipients. Suitable swelling agents include, but are not limited to, starch, modified starch or microcrystalline cellulose.

An antistatic agent can also be incorporated as a flow aid. Suitable antistatic agents include, but are not limited to, micronised or non-micronised talc, fumed silica (Aerosil™ R972), colloidal silica (Aerosil™ 200), precipitated silica (Syloid™ FP 244), and mixtures thereof.

According to one such embodiment of the invention, the granules of insulin sensitizer, sulfonylurea and/or biguanide (and optionally, one or more additional active agents such as those described herein) are characterized in that the granules are coated and comprise microcrystals of insulin sensitizer, sulfonylurea and/or biguanide, at least one binder, and optionally a diluent agent, an antistatic agent, and a coloring agent. Furthermore, the granulation excipients can also include disintegrating agents and/or surfactants.

Suitable binders include, but are not limited to, cellulosic polymers, such as ethylcellulose, hydroxypropylcellulose and hydroxypropylmethyl cellulose, acrylic polymers, such as insoluble acrylate ammoniomethacrylate copolymer, polyacrylate or polymethacrylic copolymer, povidones, copovidones, polyvinylalcohols, alginic acid, sodium alginate, starch, pregelatinized starch, sucrose and its derivatives, guar gum, polyethylene glycol, for example an acrylic polymer, such as Eudragit™ E100, and mixtures thereof.

Optionally, in order to enhance the granulation of the insulin sensitizer, sulfonylurea and/or biguanide (and one or more additional active agents), a diluent agent can be used. Suitable diluent agents include, but are not limited to, microcrystalline cellulose, sucrose, dicalcium phosphate, starches, lactose and polyols of less than 13 carbon atoms, such as mannitol, xylitol, sorbitol, maltitol, pharmaceutically acceptable amino acids, such as glycin, and their mixtures.

In one embodiment, a granule of an insulin sensitizer, a sulfonylurea and/or a biguanide (as well as any additional active agents, such as those described herein), can be in the form of a core of granulated microcrystals of insulin sensitizer, sulfonylurea and/or biguanide, coated with at least one layer comprising a further amount of an insulin sensitizer, a sulfonylurea and/or a biguanide. Such a coated core is characterized in that the core and the layer comprise each from 70% to 95%, preferably 80% to 95% by weight of insulin sensitizer, sulfonylurea and/or biguanide the balance to 100% being formed with at least one binder and optionally one or more other inactive ingredients, and that the coated core is suitably a sphere. See e.g., French patent application FR 00 14803, the disclosure of which is incorporated by reference herein.

In one embodiment of the invention, the granules can comprise (or consist essentially of): from about 10% to about 95%, e.g., from about 50% to about 70% of pioglitazone, glimepiride and/or metformin HCl and optionally one or more additional active agents, such as those described herein, at most about 20% by weight of the binder, relative to the weight of active ingredients, at most about 5%, suitably about 2% by weight of the antistatic agent, relative to the weight of said granules, suitably about 0.05% to about 5% a sweetening agent, and optionally a diluent agent for the balance to 100%.

The granules can also be coated with a coating composition comprising at least one coating polymer selected from the group consisting of cellulosic polymers, acrylic polymers and their mixtures. Among the cellulosic polymers, ethylcellulose, hydroxypropylcellulose (HPC) and hydroxypropylmethylcellulose (HPMC), can be used. Among the acrylic polymers, insoluble acrylate ammonio-methacrylate copolymer (Eudragit™ RL100 or RS100 or Eudragit RL30D or RS30D), polyacrylate (Eudragit™NE30D), or methacrylic copolymers (e.g., Eudragit™ L100-55 Eudragit™ L30D, Eudragit™ E100 and Eudragit™ EPO) can be used, alone, in combination, or in admixture with pH-dependent polymers. Eudragit™ E100 or a mixture of Eudragit™ EPO and Eudragit™ NE30D are suitably used. In one embodiment, the binder and the coating polymer are the same polymer.

Optionally, permeabilising agents, plasticizers, soluble agents, disintegrating agents and surfactants, can be added as coating additives. Suitable plasticizers include, but are not limited to, triacetine, triethylacetate, triethylcitrate (Eudraflex™), ethylphthalate, or mixtures thereof The plasticizer is used in proportions of at most about 30%, preferably 10% by weight of the coating polymers. Suitable soluble agents include polyols having less than 13 carbon atoms. Surfactants may be an anionic, nonionic, cationic, zwitterionic or amphoteric surfactant. Suitable disintegrating agents include, but are not limited to, croscarmellose, available as e.g. Ac-di-sol™, crospovidone available as e.g. Kollidon CL™, and mixtures thereof.

Suitably, the coated granules according to the present invention have a particle size distribution between about 150 μm and about 500 μm, more suitably between about 150 μm and about 425 μm, such that at least 50%, more suitably at least 70% of the granules have a particle size ranging between about 150 and about 425 μm, and less than 15% of the granules have a particle size less than about 150 μm.

In one embodiment, the coated granules according to the invention comprise: from about 10% to about 95%, preferably about 40 to about 75% of granules of one or more insulin sensitizers, one or more sulfonylureas and/or one or more biguanides, including those described herein, and optionally one or more optional additional active agents, such as those disclosed herein, sucralose from about 0.05% to about 5%, from about 5 to about 90%, suitably about 10 to about 70% and even more suitably from about 25 to about 55% of a coating polymer, such as Eudragit™ E100, the percentages being expressed by weight relative to the weight of the granules of the one or more active ingredients, from about 0 to about 10% of a permeabilising agent, such as colloidal silica, the percentages being expressed by weight relative to the weight of the coating polymer.

### Effervescent Formulations

In another embodiment, the present invention provides a solid, effervescent, rapidly dissolving dosage form of one or more of the compositions of the present invention for oral administration, such as a composition comprising one or more insulin sensitizers, one or more sulfonylureas and/or one or more biguanides, including those described herein, in the form of the formulations disclosed in U.S. Pat. No. 6,245,353, the disclosure of which is incorporated by reference herein in its entirety. In such an embodiment, the effervescent formulation comprises (or consists essentially of) (a) a therapeutic amount of each of one or more insulin sensitizers, one or more sulfonylureas and/or one or more biguanides, including those described herein, (b) an effervescent base comprising at least one of (i) at least one of (1) an organic edible acid and (2) a salt thereof, (ii) at least one of an alkali metal and an alkaline earth metal carbonate and bicarbonate, and (c) optionally pharmaceutically acceptable auxiliary ingredient. The amounts of one or more insulin sensitizers, one or more sulfonylureas and/or one or more biguanides, including those described herein, other optional active agents, and other ingredients in the effervescent formulations of the present invention are readily determinable by those of ordinary skill in the art, and include those amounts and combinations described herein.

For use of the effervescent dosage forms of this aspect of the invention, a solution or suspension of one or more insulin sensitizers, one or more sulfonylureas and/or one or more biguanides, including those described herein, is formed by adding water to the soluble or dispersible effervescent tablets or soluble granules, with evolution of CO₂ gas. The resulting effervescent solution or suspension can be ingested very easily, even by patients who have difficulties swallowing. The rapidly disintegrating tablet can also be administered so that it directly disintegrates in the mouth. A rapid release of the active ingredient(s) is of particular importance here, to ensure a rapid onset of action.

Effervescent agents capable of releasing CO₂, which can be used in the present invention, include alkali metal carbonates or alkali metal bicarbonates, such as sodium carbonate or sodium bicarbonate. Agents for inducing CO₂ release which are suitably employed are edible organic acids, or their acidic salts, which are present in solid form and which can be formulated with the one or more active ingredients of the compositions of the present invention and the other auxiliary ingredients (as well as any other active agents) to provide granules or tablets, without premature evolution of CO₂. Edible organic acids which can be so used include for example, tartaric acid, malic acid, fumaric acid, adipic acid, succinic acid, ascorbic acid, maleic acid or citric acid. Pharmaceutically acceptable acidic salts include, for example, salts of polybasic acids which are present in solid form and in which at least one acid function is still present, such as sodium dihydrogen or disodium hydrogen phosphate or monosodium or disodium citrate.

In one embodiment, the present invention provides effervescent pharmaceutical formulations including the formulations and compositions described herein, having an effervescent base comprising (a) a mixture of calcium carbonate with an organic edible acid; (b) a mixture of calcium carbonate, sodium carbonate, sodium bicarbonate and an organic edible acid; or (c) a mixture of sodium bicarbonates, sodium carbonate and an organic edible acid.

The soluble or dispersible effervescent tablets or the soluble granules of this aspect of the invention suitably comprise (or consist essentially of) from about 2-120 mg of an insulin sensitizer, from about 0.5-40 mg of a sulfonylurea, from about 200-5000 mg of a biguanide, and from about 50 mg to about 5000 mg, suitably from about 500 mg to about 3000 mg, of an effervescent base, optionally along with one or more other active agents (such as those described herein) and excipients suitably at about 0.05% to about 5%.

The effervescent base suitably comprises from about 100 mg to about 500 mg calcium ions as, for example, calcium carbonate, and from about 20 mg to about 1500 mg citric acid and/or its salts. In another embodiment, the effervescent base comprises from about 50 mg to about 2000 mg sodium bicarbonate, from about 20 mg to about 200 mg of sodium carbonate and from about 20 mg to about 1500 mg citric acid and/or from about 20 mg to about 500 mg tartaric acid.

An additional suitable composition of the effervescent base comprises from about 50 mg to about 500 mg sodium bicarbonate, from about 20 mg to about 100 mg sodium carbonate, and from about 50 mg to about 750 mg calcium carbonate and from about 100 mg to about 1500 mg of citric acid.

The soluble/dispersible tablets can be prepared by known processes for preparing effervescent bases, such as those disclosed in U.S. Pat. No. 6,245,353, the disclosure of which is incorporated herein by reference in its entirety.

### Orally Dissolving/Consumable Films

Another embodiment of the present invention is directed to a physiologically acceptable film that is particularly well-adapted to dissolve in the oral cavity of a warm-blooded animal including humans, and adhere to the mucosa of the oral cavity, to allow delivery of one or more of the pharmaceutical combination formulations of the present invention, such as a formulation comprising one or more insulin sensitizers, one or more sulfonylureas and/or one or more biguanides, including those described herein, and optionally one or more additional active agents, such as those described herein. Such physiologically acceptable films suitable for use in accordance with this aspect of the present invention are disclosed in U.S. Patent Application No. 2004/0247648, the disclosure of which is incorporated herein by reference in its entirety.

In one such embodiment of the present invention, an orally dissolving/consumable film comprises a modified starch, one or more insulin sensitizers, one or more sulfonylureas and/or one or more biguanides, optionally one or more additional active agents such as those described herein, and optionally, at least one water soluble polymer. The amounts of one or more insulin sensitizers, one or more sulfonylureas and/or one or more biguanides, other optional active agents in the orally dissolving/consumable film formulations of the present invention are readily determinable by those of ordinary skill in the art, and include those amounts and combinations described herein.

The consumable films of the present invention may comprise one or more of the following ingredients: water, antimicrobial agents, additional film forming agents or water soluble polymers, plasticizing agents, flavorings, sulfur precipitating agents, saliva stimulating agents, cooling agents, surfactants, stabilizing agents, emulsifying agents, thickening agents, binding agents, coloring agents, triglycerides, polyethylene oxides, propylene glycols, additional taste-masking agents or sweeteners, fragrances, preservatives and the like, as described in U.S. Pat. No. 6,596,298, the disclosure of which is incorporated by reference herein in its entirety.

In one such embodiment, the consumable films of the present invention include a modified starch. The modified starches used in accordance with the present invention can be prepared by mechanically, chemically or thermally modifying unmodified starches. For example, modified starches may be prepared by chemically treating starches to produce, for example, acid treatment starches, enzyme treatment starches, oxidized starches, cross-bonding starches, and other starch derivatives. Starches suitable for modification to produce modified starches may be obtained from natural products such as corn, potatoes, tapioca as well as genetically modified forms of the same such as high amylose and waxy corn as well as sorghum varieties.

Examples of modified starches for use in the practice of the present invention include, but are not limited to, modified corn starches, modified tapioca starches, acid and enzyme hydrolyzed corn and/or potato starches, hypochlorite-oxidized starches, acid-thinned starches, ethylated starches, cross-bonded starches, hydroxypropylated tapioca starches, hydroxypropylated corn starches, pregelatinized modified starches, and the like. Preferred modified starches are selected from pregelatinized modified corn starches and pregelatinized modified tapioca starches.

Representative examples of commercially available modified starches useful in the present invention include PURE-COTE™ modified starches such as PURE-COTE™ B793 (a pregelatinized modified corn starch) and PURE-COTE™ B795 (a pregelatinized modified corn starch), for example, available from Grain Processing Corporation, 1600 Oregon Street, Muscatine, Iowa 52761-1494 USA.

In one such embodiment of the present invention, the modified starch is present in amounts ranging from about 1% to about 90% by weight, in another embodiment about 10% to about 90% by weight, and in yet another embodiment from about 35% to about 80% by weight of the film.

Modified starch may be included in the film alone or optionally in combination with an additional water soluble film forming polymers such as those selected from, for example, pullulan, hydroxypropylmethyl cellulose, hydroxyethyl cellulose, hydroxypropyl cellulose, polyvinyl pyrrolidone, carboxymethyl cellulose, polyvinyl alcohol, sodium alginate, polyethylene glycol, tragacanth gum, guar gum, acacia gum, arabic gum, polyacrylic acid, methylmethacrylate copolymers, carboxyvinyl polymers, amylose, high amylose starch, hydroxypropylated high amylose starch, pectin, dextrin, chitin, chitosan, levan, elsinan, collagen, gelatin, zein, gluten, soy protein isolate, whey protein isolate, casein and combinations thereof. A preferred water soluble polymer is pullulan. The amount of the water soluble polymer typically is up to about 99% by weight, suitably up to about 80% by weight, more suitably up to about 50% by weight, and most suitably up to about 40% by weight of the film.

### Liquid Dosage Forms

In some embodiments, the compositions of the invention are tormulated into liquid dosage forms that are suitable for oral administration including pharmaceutically acceptable emulsions, solutions, suspensions, syrups and elixirs. In addition to the active compound(s), the liquid dosage forms may contain inert diluents and/or solvents commonly used in the art. Water is the solvent of choice for the formulations of the invention; however, combinations of water with other physiologically acceptable solvents as required are also satisfactory for use. Other solvents, solubilizing agents and emulsifiers suitable for use in place of, or in addition to, water include but are not limited to saturated aliphatic mono- and polyvalent alcohols which contain 2-6 carbon atoms (including, but not limited to, ethanol, 1,2-propylene glycol, sorbitol, and glycerine), polyglycols such as polyethylene glycols, and surfactants/emulsifiers like the fatty acid esters of sorbitan, and mixtures thereof. Oils, in particular, cottonseed, peanut, or corn oils, may also be added to the compositions. The combination of the additional solvents in the aqueous solution should preferably not exceed about 15% (w/v) of the total composition. Besides inert diluents, the oral compositions can also include adjuvants such as wetting agents, emulsifying and suspending agents (e.g., microcrystalline cellulose, sodium carboxymethyl cellulose, hypromellose, carbopol and the like), surfactants, sweetening, flavoring, and perfuming agents, including those described in further detail herein below. Liquid dosage forms that provide the active ingredient(s) in suspension may comprise, in addition to the active compound(s), one or more suspending agents such as microcrystalline cellulose, magnesium aluminum silicate, bentonite, agar-agar, hypromellose, sodium carboxymethyl cellulose, carbopol/carbomer, pectin, acacia, tragacanth or their mixtures.

### Sustained-Release/Extended-Release Formulations and Dosage Forms

In certain embodiments of the invention described herein, one or more of the active pharmaceutical ingredients contained in the present compositions are present in an immediate-release form. In additional embodiments of the invention, one or more of the active pharmaceutical ingredients contained in the present compositions, and suitably the metformin or pharmaceutically acceptable salt or ester thereof (e.g., metformin HCl), is present in such formulations in a sustained-release or extended-release form. As a practical matter, the phrases “immediate-release,” “sustained-release” and “extended-release” are terms of art, the definitions of which will be readily familiar to those of ordinary skill in the relevant arts in view of knowledge that is readily available and well-known in those arts, particularly in medicine, pharmaceutical formulations and pharmacology. Suitable such embodiments will optimally comprise a therapeutically effective amount or dose of one or more of the active pharmaceutical ingredients, suitably metformin or a pharmaceutically acceptable salt or ester thereof, and one or more pharmaceutically acceptable carriers or excipients that provide a sustained or extended release of the one or more active pharmaceutical ingredients over a period of time after administration to the patient. In certain such embodiments, the one or more active pharmaceutical ingredients, suitably metformin or a pharmaceutically acceptable salt or ester thereof, is: 1) coated with one or more sustained release components; 2) bound to a cation exchanger; 3) reacted with one or more osmotically active substances and coated with a semi-permeable membrane and a hole is bored into the membrane; or 4) embedded in, or is bound to, one or more substances selected from of the group consisting of digestible fats, indigestible fats, polymers and swelling agents. In certain such embodiments, the metformin of the present invention will be either in an inner core coated with a preparation of insulin sensitizer and glimepiride, or in a second layer apart from the layer containing the insulin sensitizer and glimepiride.

Methods for preparing sustained release tablets, capsules, caplets, pellets and the like, as well as excipients for use in the sustained release formulations of the present invention, are well known in the art, and can be found, for example, throughout the detailed description section and the Examples of U.S. Pat. No. 5,271,946, the disclosure of which is incorporated herein by reference in its entirety for all purposes.

As discussed in U.S. Pat. No. 5,271,946, the sustained release formulations of the present invention can be obtained as follows:

1. Through binding of one or more of the active ingredients contained in the present compositions, suitable metformin or a pharmaceutically acceptable salt or ester thereof (e.g., metformin HCl), and optionally one or more additional active agents such as those described herein, to physiologically acceptable cation exchangers. The following may, for example, be used as such cation exchangers: acrylic and methacrylic resins with exchangeable protons, acid groups: COO⁻ e.g. Amberlite™ IRP-64 Polystyrene resins with exchangeable Na⁺, acid groups: SO₃⁻, e.g. Amberlite™ IRP-69.

2. Coating of active ingredient particles, granulate or pellet grains or active ingredient-containing tablets with coatings of the following substances, or mixtures of the following substances: hydroxypropylmethyl cellulose phthalate- or acetate succinate; cellulose-, starch-, as well as polyvinyl acetate phthalate; carboxymethyl cellulose; hypromellose; carbopol starch acetate; cellulose acetate; polyvinyl acetate; methylcellulose phthalate, methylcellulose succinate, methyl cellulose phthalate succinate as well as methyl cellulose phthalic acid half ester; zein; ethyl cellulose as well as ethyl cellulose succinate; shellac; gluten; ethylcarboxyethyl cellulose; ethacrylate-maleic acid anhydride copolymer; maleic acid anhydride vinyl methyl ether copolymer; sterol maleic acid copolymerizate; 2-ethylhexylacrylate maleic acid anhydride; crotonic acid vinyl acetate copolymer; glutaminic acid/glutaminic acid ester copolymer; carboxymethylethyl cellulose glycerin mono-octanoate; cellulose acetate succinate; polyarginin; fats, oils, waxes, fatty alcohols; anionic polymerizates of methacrylic acid and methacrylic acid esters (Eudragit™L, Eudragit™S); copolymerizates of acrylic and methacrylic acid esters with a low ammonium group (Eudragit™RS) content, as well as copolymers of acrylic and methacrylic acid esters and trimethyl ammonium methacrylate (Eudragit™RL), copolymerizates of acrylic acid ethyl- and methacrylic acid methyl esters 70:30 (Eudragit™NE 30 D), copolymerizates of acrylic acid, methacrylic acid as well as their esters (ratio of the free carboxyl groups to the ester groups for example 1:1) (Eudragit™L 30 D).

Such sustained release formulations may also contain conventional softeners (e.g. dibutyl sebacate, citric and tartaric acid esters, glycerin and glycerin esters, phthalic acid esters and similar substances). It also is possible to add water-soluble substances such as polyethylene glycols, polyvinylpyrrolidone, copolymerizates of polyvinylpyrrolidone and polyvinyl acetate, hydroxypropyl cellulose, hydroxypropylmethyl cellulose. The addition of solids such as talcum and/or magnesium stearate to the coating is also possible.

Organic acids (such as for example citric acid, tartaric acid, maleic, fumaric, ascorbic acid) may also be incorporated into the pellet grains, granulate grains or tablets.

3. Coating of pressed disks, tablets, granulates containing the active ingredient(s), suitably metformin or salt/ester thereof, and optionally one or more additional active agents such as those described herein, and one or more osmotically active substances, (e.g. mannitol, sorbitol and the like) with a semi-permeable membrane, e.g. of 70 to 90 weight % of cellulose acetate and hydroxypropylmethyl cellulose or hypromellose (30 to 10 weight %).

Other osmotically active substances that can be used include organic and inorganic compounds or soluble substances which generate an osmotic pressure gradient as compared to the outer liquid via the semi-permeable wall. Osmotically active agents or osmotically active compounds include magnesium sulfate, magnesium chloride, sodium chloride, lithium chloride, potassium sulfate, potassium hydrogen phosphate, urea, saccharose and the like. Other osmotically active agents are disclosed in U.S. Pat. Nos. 3,854,770, 4,077,407 and 4,235,236, the disclosures of each of which are incorporated herein by reference in their entireties.

Semi-permeable materials which can be used as polymers for osmosis and reverse osmosis are, for example: cellulose acylate, cellulose diacylate, cellulose triacylate, cellulose acetate, cellulose diacetate, cellulose triacetate, β-glucan acetate, acetaldehyde dimethyl acetate, cellulose acetate ethyl carbamate, polyamide, polyurethane, sulphonated polystyrene, cellulose acetate phthalate, cellulose acetate methyl carbamate, cellulose acetate succinate, cellulose acetate dimethylamino acetate, cellulose acetate chloracetate, cellulose dipalmitate, cellulose dioctanoate, cellulose dicaprylate, cellulose dipentanate, cellulose acetate valerate, cellulose acetate-p-toluene sulphonate, cellulose acetate butyrate, ethyl cellulose, selectively permeable polymers which are formed by joint precipitation of a polycation and a polyanion as disclosed in U.S. Pat. Nos. 3,173,876, 3,276,586, 3,541,005, 3,541,006 and 3,546,142, the disclosures of which are incorporated by reference herein in their entireties. Coatings of this type in semi-permeable membranes may for example also be effected according to U.S. Pat. Nos. 4,455,143 and 4,449,983, the disclosures of which are incorporated by reference herein.

The proportion of osmotically active substance can be from about 10 to about 800 parts by weight, suitably about 20 to about 600, and more suitably about 50 to about 400 parts by weight, based on 1 part by weight of active ingredient that is to be sustainably or extendedly released. The amount of coating substances applied is such that the semi-permeable membrane is about 50 to about 500 μm, suitably about 100 to about 300 μm thick.

4. Embedding of or binding of one or more active ingredients in the present compositions, suitably metformin (or salt/ester thereof), and/or any other optional additional active agent(s) to the following substances or mixtures of these substances:

Digestible fats, such as triglycerides of saturated fatty acids, C₈14₁₆O₂ to C₁₈H₃₆O₂, and mixtures thereof, peanut oil and hydrated peanut oil, castor oil and hydrated castor oil, olive oil, sesame oil, cottonseed oil and hydrogenated cottonseed oil, corn oil, wheat germ oil, sunflower seed oil, cod liver oil, mixtures of mono-, di- and triesters of palmitic and stearic acid with glycerine, glycerine trioleate, diglycol stearate, stearic acid.

Indigestible fats or fat-like substances, for example esters of aliphatic saturated or unsaturated fatty acids (2 to 22 carbon atoms, in particular 10 to 18 carbon atoms) with monovalent aliphatic alcohols (1 to 20 carbon atoms), carnauba wax, beeswax, fatty alcohols (straight chain or branched) of chain length C₈H₁₇OH to C₃₀H₆₁OH, in particular C₁₂H₂₅OH to C₂₄H₄₉OH.

Polymers such as polyvinyl alcohol, polyvinyl chloride, polyacrylic acid (Carbopol™); anionic polymerizates of methacrylic acid and methacrylic acid esters (Eudragit™L, Eudragit™S), acrylic and methacrylic acid ester copolymerizates with trimethyl ammonium methacrylate (Eudragit™RL, Eudragit™RS).

Copolymerizates of ethyl acrylates and methyl methacrylates (Eudragit™NE 30 D), as well as of acrylic acid, methacrylic acid as well as esters thereof (ratio of free carboxyl groups to ester groups 1:1) (Eudragit™L 30 D), polyethylene, polyglycolic acid, polyhydroxybutyric acid, polylactic acid, copolymers of lactic acid and glycolic acid (manufacturer: Boehringer Ingelheim), copolymers of lactic acid and ethylene oxide, copolymers of glycolic acid and ethylene oxide, copolymers of lactic acid and hydroxybutyric acid, hydroxypropylmethyl cellulose-phthalate or -acetate succinate; cellulose acetate phthalate, starch acetate phthalate as well as polyvinyl acetate phthalate; carboxymethyl cellulose; methylcellulose phthalate, -succinate, -phthalate succinate, methyl cellulose phthalic acid half ester; zein; ethyl cellulose; shellac, gluten; ethylcarboxyethyl cellulose; ethacrylate maleic acid anhydride copolymer; maleic acid anhydride vinyl methyl ether copolymer; styrene maleic acid copolymerizate; 2-ethylhexyl acrylate maleic acid anhydride; crotonic acid vinyl acetate copolymer; glutaminic acid/glutaminic acid ester copolymer; carboxymethyl cellulose glycerine mono-octanoate; cellulose acetate succinate; polyarginine; cross-linked alginate; cross-linked gelatin.

Swelling agents such as methyl cellulose, hydroxypropyl cellulose, hydroxypropyl methyl cellulose (Pharmacoat, Methocel E (propylene glycol ether of methyl cellulose)), alginic acid and their salts (Na⁻, Ca⁻ salt, also mixtures of sodium alginate and calcium salts such as CaHPO₄), starch, carboxymethyl starch, carboxymethyl cellulose and their salts (e.g. Na⁻ salts), galacto mannan, gum arabic, karaya rubber, ghatti gum, agar-agar, carrageen, xanthan rubber, guar rubber and its derivatives, carob bean flour, propylene glycol alginate, pectin, tragacanth.

The amounts of active ingredient(s), suitably metformin or salt/ester thereof, and optionally one or more additional active agents such as those described herein, in the formulations of the invention are the same as those described elsewhere herein for non-sustained-release (i.e., “immediate release” or “delayed release”) dosage forms of the compositions of the invention.

Suitable exemplary sustained release components are:

(a) Cation exchangers: Sodium poly(styrene, divinylbenzene)sulphonate (e.g. Amberlite™IRP 69). Suitably 3 to 10 parts of Amberlite™IRP 69 are for example used per 1 part of active pharmaceutical ingredient.

(b) Coating substances: Hydroxypropylmethyl cellulose phthalate, suitably at 1.5 to 3 parts of hydroxypropyl methyl cellulose phthalate 55 are used per 1 part of active ingredient, e.g., one or more insulin sensitizers, one or more sulfonylureas and/or one or more biguanides. Ethyl cellulose, suitably 0.1 to 1 part of ethyl cellulose are used per 1 part of active ingredient. Eudragit resins, for example Eudragit™RS 0.01 to 0.1 part of Eudragit™RS per 1 part of active pharmaceutical ingredient.

(c) Semi-permeable layers with osmotically acting active substance containing core and outlet openings: Coating with 100 to 300 μm thick layer of 82% cellulose acetate and 18% hydroxypropyl methyl cellulose.

(d) Embedding substances: Hydrocolloids e.g. hydroxypropyl methyl cellulose: 2 to 10 parts of hydrocolloid per 1 part of active ingredient. Eudragit™RS: 10 to 15 parts of Eudragit™RS per 1 part of active ingredient. Glycerineditripalmito stearate (e.g. Precirol Ato 5) 1 to 10 parts of Precirol Ato 5 per 1 part of active pharmaceutical ingredient.

The requisite release of one or more of the active pharmaceutical ingredients included in the present formulations (e.g., one or more insulin sensitizers, one or more sulfonylureas and one or more biguanides, e.g., metformin, suitably metformin HCl, and optionally any additional active agents) of 0.5 to 200 mg per hour suitably occurs within the desired range through the parameters described herein. Should it be desired to achieve a specific release rate within this range it is possible, for example, to proceed as follows:

1. The preparation of the coating or embedding of the active substance in the described manner.

2. Testing of the release of active substance from the dosage form using 0.1 N HCl (2 hours) and phosphate buffer pH 6.8 (subsequently) as release medium.

3. (a) Should too much substance be released: Increase of the proportion of the sustained release component and/or reduction of the proportion of water-soluble auxiliary substances. Reduction of the proportion of osmotically active substance.

(b) Should too little substance be released: Reduction of the proportion of the sustained release component and/or increase of the proportion of water soluble auxiliary substances. Increase of the proportion of osmotically active substance.

In one embodiment, a release rate of about 20 mg to about 200 mg of metformin HCl per hour can be achieved, allowing the use of metformin HCl at doses as low as 500 mg/day but up to about 5000 mg/day if desired.

### Methods

In certain embodiments, the present invention provides methods of treating or preventing glycemic imbalance disorders in a mammal. Such disorders include, but are not limited to, type II diabetes mellitus, type I diabetes, diabetes insipidus, hyperglycemia, hypothyroidism, and the like. Certain such methods comprise administering to said mammal glimepiride (a sulfonylurea), metformin (a biguanide) and an insulin sensitizer thiazolidinedione (a glitazone). In certain such embodiments, the methods comprise administering these three active ingredients (i.e., a sulfonylurea such as glimepiride, a biguanide such as metformin (or metformin hydrochloride), and an insulin sensitizer such as a glitazone, e.g., pioglitazone or rosiglitazone). Hence, the present invention further provides methods for reducing glucose levels in blood and preventing the development of diabetes in a mammal, comprising administering to said mammal one or more of the composition(s) of the present invention.

In some embodiments, the invention provides methods of administering a pharmaceutical composition comprising glimepiride, pioglitazone, and metformin. In some embodiments, the invention provides methods of administering a pharmaceutical composition comprising glimepiride, rosiglitazone, and metformin. In some embodiments, the metformin is formulated as immediate-release within the dosage form. In other embodiments, the metformin is formulated as sustained-release within the dosage form.

In some embodiments, the glimepiride of the present invention will administered at a daily dose of about 0.5 mg/day to about 40 mg/day, about 1 mg/day to about 10 mg/day, or about 2 mg/day to about 6 mg/day. In some embodiments, the glimepiride of the present invention is administered at a dose of 2 mg/day.

In some embodiments, the insulin sensitizer of the present invention will be administered at doses from about 2 mg/day to about 120 mg/day, about 10 mg to about 80 mg/day, about 15 mg/day to about 60 mg/day, most preferably at a dose of 15 mg/day.

In some embodiments, the metformin of the present invention will be administered at doses of about 500 mg/day to about 5000 mg/day, or about 500 mg/day to about 3000 mg/day. In some embodiments, the metformin will be administered at a dose of about 850 mg/day.

In some embodiments, the methods comprising administering to a mammal a pharmaceutical composition(s) of the present invention that is in a solid or a liquid formulation.

In some embodiments, the pharmaceutical formulation of the present invention is administered daily for a shorter duration, e.g., 1 week to 12 weeks (week 1 to week 12). In some embodiments, the HbA1c levels in a subject are reduced by about 4%, about 3% to about 5%, about 2% to about 6%, or about 5% to about 10% on week 12. In some embodiments, the mean decrease in fasting blood glucose levels (FBG) (mg/dL) are reduced by about 20% to about 90%, about 50% to about 85%, about 60% to about 80% on week 12.

In some embodiments, the cholesterol levels in a subject are reduced by about 10 to about 30 mg/dL within 24 weeks using the compositions and methods of the present invention as described herein. in some embodiments, the triglyceride levels in a subject are reduced by about 20 to about 50 mg/dl within 24 weeks using the compositions and methods of the present invention as described herein.

In some embodiments, the methods of the present invention can include administration of the dosage form daily for extended periods of time, e.g., about 6 months, about 1 year, about 18 months, about 2 years, about 5 years, about 10 years, about 20 years, or indefinitely for the duration of a subject's life. The method also can include administration of the dosage form daily for shorter periods of time, e.g., once daily for at least 7, 14, 21, or 28 consecutive days.

Type II diabetes is associated with high HbA1c levels and high FBG levels. Thus, in some embodiments, the present invention is directed to methods of reducing HbA 1c levels in a subject, the methods comprising administration the pharmaceutical formulations of present invention. In some embodiments, the present invention is directed to methods of reducing FBG levels in a subject, the methods comprising administration the pharmaceutical formulations of present invention. The invention is also directed to methods of reducing, preventing, or slowing the development of diabetes in humans comprising administration of the pharmaceutical formulations of the present invention. The term “preventing” means to stop or hinder a disease, disorder, or symptom of a disease or condition.

The term “subject” refers to mammals such as humans or primates, such as apes, monkeys, orangutans, baboons, gibbons, and chimpanzees. The term “subject” can also refer to companion animals, e.g., dogs and cats; zoo animals; equids, e.g., horses; food animals, e.g., cows, pigs, and sheep; and disease model animals, e.g., rabbits, mice, and rats. The subject can be a human or non-human. The subject can be of any age. For example, in some embodiments, the subject is a human infant, i.e., post natal to about 1 year old; a human child, i.e., a human between about 1 year old and 12 years old; a pubertal human, i.e., a human between about 12 years old and 18 years old; or an adult human, i.e., a human older than about 18 years old. In some embodiments, the subject is an adult, either male or female.

In some embodiments, the subject is a “subject in need thereof.” A subject in need thereof refers to an individual for whom it is desirable to treat, i.e., to reduce blood glucose levels, prevent high blood glucose levels, or retard the onset of diabetes.

“Pharmaceutically acceptable” refers to compositions that are, within the scope of sound medical judgment, suitable for contact with the tissues of human beings and animals without excessive toxicity or other complications commensurate with a reasonable benefit/risk ratio. In some embodiments, the compounds, compositions, and dosage forms of the present invention are pharmaceutically acceptable.

In some embodiments, the pharmaceutical compositions of the present invention are administered continuously. The term “continuous” or “consecutive,” as used herein in reference to “administration,” means that the frequency of administration is at least once daily. Note, however, that the frequency of administration can be greater than once daily and still be “continuous” or “consecutive,” e.g., twice or even three times daily.

Administration of the pharmaceutical compositions of the present invention can be achieved using various regimens. For example, in some embodiments, administration of the pharmaceutical compositions of the present invention is daily on consecutive days, or alternatively, the dosage form is administered every other day (bi-daily). Administration can occur on one or more days. For example, in some embodiments the pharmaceutical compositions of the present invention are administered daily for the duration of the subject's lifetime, or from 1 year to 20 years or 5 years to 10 years. In some embodiments, administration of the pharmaceutical compositions of the present invention occurs for 7, 14, 21, or 28 days. In some embodiments, administration of the pharmaceutical compositions of the present invention occurs until the blood glucose levels of the subject are lowered to a preselected target level, the target level being determined by a medical professional. In some embodiments, administration of the pharmaceutical compositions of the present invention continues even after the blood glucose levels of the subject have reached normal or borderline levels, or to a preselected target level. In some embodiments, the administration of the pharmaceutical compositions of the present invention is done as a prophylactic measure, before blood glucose levels, glycated HbA1c levels, fasting blood glucose levels, etc. become elevated, and before post-prandial glycemia (a requirement for insulin administration) develops.

Administration of the dosage forms of the present invention can be combined with other regimens used to treat type II diabetes mellitus and related disorders. For example, the method of the present invention can be combined with diet regimens (e.g., low carbohydrate diets), exercise regimens, or weight loss regimens to reduce blood sugar levels. The methods of the present invention can also be used in combination with other pharmaceutical products to reduce blood sugar levels in a subject. Other regimens can also include other blood sugar-lowering pharmaceutical products including, e.g., α-glucosidase inhibitors, meglitinides, and dipeptidyl peptidase-IV inhibitors.

The present invention is directed to kits or packages containing one or more dosage forms to be administered according to the methods of the present invention. A kit or package can contain one dosage form, or more than one dosage forms (i.e., multiple dosage forms). If multiple dosage forms are present in the kit or package, the multiple dosage forms can be optionally arranged for sequential administration. The kits can contain dosage forms of a sufficient number to provide convenient administration to a subject who has a chronic condition and requires long-term administration of the pharmaceutical formulations of the present invention. Each dosage form can be intended for ingestion on successive days. For example, in some embodiments, the kit provides dosage forms of a sufficient number for 1, 2, 3 or 4 months of daily administration of the pharmaceutical formulations of the present invention. In some embodiments of the present invention, the kit comprises dosage forms for shorter periods of administration, e.g., the kit can contain about 7, 14, 21, 28 or more dosage forms for oral administration, each dosage form intended for ingestion on successive days.

The kits of the present invention can optionally contain instructions associated with the dosage forms of the kits. Such instructions can be in a form prescribed by a governmental agency regulating the manufacture, use or sale of pharmaceutical products, which notice reflects approval by the agency of the manufacture, use or sale for human administration to treat a condition or disorder. The instructions can be in any form which conveys information on the use of the dosage forms in the kit according to the methods of the invention. For example, the instructions can be in the form of printed matter, or in the form of a pre-recorded media device.

In the course of examination of a patient, a medical professional may determine that administration of one of the methods of the present invention is appropriate for the patient, or the physician may determine that the patient's condition can be improved by the administration of one of the methods of the present invention. Prior to prescribing a regimen of administering the pharmaceutical formulations of the present invention, the physician can counsel the patient, for example, on the various risks and benefits associated with the regimen. The patient can be provided full disclosure of all the known and suspected risks associated with the regimen. Such counseling can be provided verbally, as well as in written form. In some embodiments, the physician can provide the patient with literature materials on the regimen, such as product information, educational materials, and the like.

A “medical professional,” includes, for example, a physician, physician assistant, nurse practitioner, pharmacist and customer service representative. All of the various aspects, embodiments and options described herein can be combined in any and all variations.

It will be readily apparent to one of ordinary skill in the relevant arts that other suitable modifications and adaptations to the methods and applications described herein may be made without departing from the scope of the invention or any embodiment thereof It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the claims. Having now described the present invention in detail, the same will be more clearly understood by reference to the following examples, which are included herewith for purposes of illustration only and are not intended to be limiting of the invention.

### EXAMPLES

### Example 1

### Therapeutic Effects of Pioglitazone/Glimepiride Combination Therapy on Type II Diabetes

Objective: The study was designed to test the effect of combination therapy with pioglitazone and glimepiride on glycated hemoglobin (HbA1c) and fasting blood glucose (FBG) in patients with type 2 diabetes inadequately controlled by diet alone.

Research Design and Methods: The study was a 12-week, double blind, randomized, parallel group, multi-center study, with 120 patients randomized to receive pioglitazone (15 mg per day), glimepiride (2 mg per day), or a combination of pioglitazone plus glimepiride (15 mg per day; 2 mg per day). Patients started a strict dietary regimen 1 week before receiving the first dose of each treatment. The study was open to patients with type 2 diabetes mellitus and having HbA1c levels greater than 8%. Patients were over 30 years of age and have had diabetes for at least 1 year.

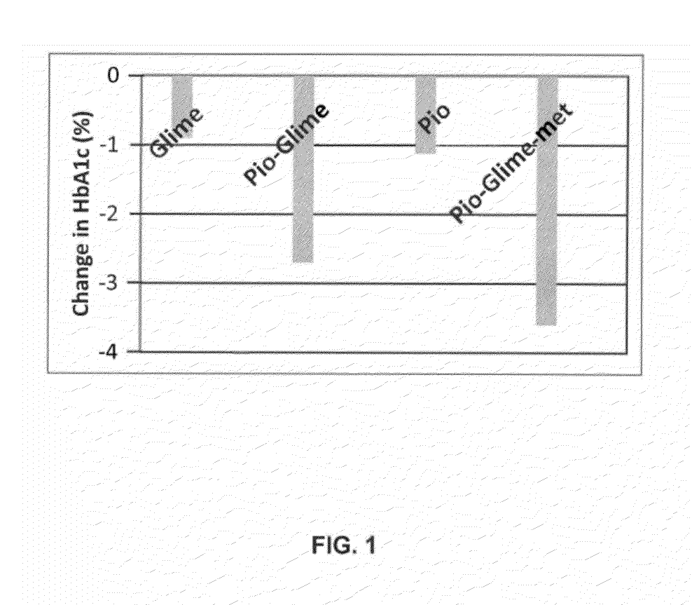

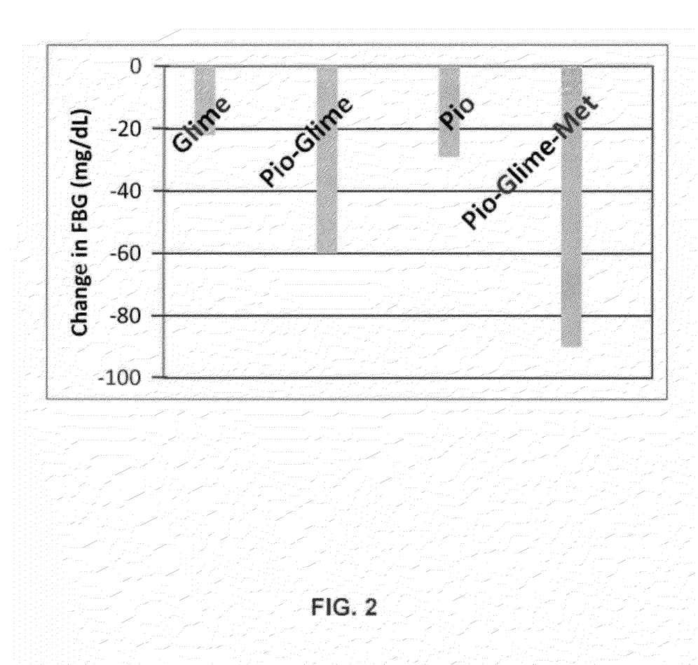

Results: The primary end point, HbA1c levels, decreased from baseline in all three groups after 12 weeks. The decrease was greater in the group receiving combined therapy compared with other two monotherapies (−2.7% in combined therapy group, −0.14% in pioglitazone group and −0.98% in the glimepiride group, FIG. 1). Combination therapy was more effective than either monotherapy in reducing fasting blood glucose (−60 mg/dL in combined therapy, −28 mg/dL in pioglitazone group and −22 mg/dL in glimepiride group; FIG. 2). After 3 months of intervention, about 50% of patients undergoing combined therapy had attained adequate glycemic control (HbA1c levels of less than 7%).

Patients having HbA1c levels greater than 7% have a high risk for developing cardiovascular disease and other complications of diabetes. Thus, in these patients, it is necessary to add a third agent to the regimen in order to obtain HbA1c values below 7%. Based upon a meta-analysis of metformin clinical trials, the addition of metformin as a third agent to this regimen will produce an additional reduction in HbA1c of about 1% and will contribute to adequate glycemic control in such patients with advanced type 2 diabetes. The addition of metformin in this triple therapy provides a synergistic mechanism of action, both improving insulin sensitivity and suppressing hepatic glucose output.

All of the various embodiments or options described herein can be combined in any and all variations. While the invention has been particularly shown and described with reference to some embodiments thereof, it will be understood by those skilled in the art that they have been presented by way of example only, and not limitation, and various changes in form and details can be made therein without departing from the spirit and scope of the invention. Thus, the breadth and scope of the present invention should not be limited by any of the above described exemplary embodiments, but should be defined only in accordance with the following claims and their equivalents.

All documents cited herein, including journal articles or abstracts, published or corresponding U.S. or foreign patent applications, issued or foreign patents, or any other documents, are each entirely incorporated by reference herein, including all data, tables, figures, and text presented in the cited documents.

What is claimed is:

1. A pharmaceutical composition suitable for oral administration for the treatment of diabetes and related disorders, comprising at least one insulin sensitizer, glimepiride, and metformin or pharmaceutically active salt or ester thereof

2. The oral formulation according to claim 1, wherein the oral formulation is a solid formulation.

3. The oral formulation according to claim 2, wherein the solid formulation is selected from the group consisting of capsules, tablets, pills, powders, particles, and granules.

4. The oral formulation according to claim 1, wherein the oral formulation is a liquid formulation.

5. The oral formulation according to claim 4, wherein the liquid formulation is selected from the group consisting of emulsions, solutions, suspensions, syrups, and elixers.

6. The oral formulation according to claim 1, wherein the oral formulation comprises from about 0.5 mg to about 40 mg of glimepiride.

7. The oral formulation according to claim 1, wherein the oral formulation comprises from about 2 mg to about 120 mg of said insulin sensitizer.

8. The oral formulation according to claim 1, wherein the oral formulation comprises from about 500 mg to about 5000 mg of metformin hydrochloride.

9. The oral formulation according to claim 1, wherein the oral formulation comprises sustained-release or extended-release metformin hydrochloride.

10. The oral formulation according to claim 1, wherein the insulin sensitizer is a glitazone.

11. The oral formulation of claim 10, wherein said glitazone is pioglitazone.

12. The oral formulation according to claim 10, wherein said glitazone is rosiglitazone.

13. The oral formulation according to claim 1, wherein said formulation comprises about 2 mg to about 120 mg of an insulin sensitizer selected from the group consisting of pioglitazone and rosiglitazone, about 0.5 mg to about 40 mg glimepiride, and about 500 mg to about 5000 mg metformin hydrochloride.

14. A kit for use in treating Type II diabetes, comprising the oral formulation of claim 1 and a label containing instructions to administer the oral formulation once per day, twice per day, three times per day, or four times per day to a subject in need thereof

15. A method for treating diabetes and related disorders in a mammal, comprising administering to said mammal an oral formulation comprising at least one insulin sensitizer, glimepiride, and metformin or pharmaceutically active salt or ester thereof,

16. The method according to claim 15, wherein said mammal is human.

17. The method according to claim 15, wherein the method comprises administering to said mammal a daily dose of glimepiride of about 0.5 mg to about 40 mg.

18. The method according to claim 15, wherein the method comprises administering to said mammal a daily dose of said insulin sensitizer of about 2 mg to about 120 mg.

19. The method according to claim 15, wherein the method comprises administering to said mammal a daily dose of metformin, or pharmaceutically active salt or ester thereof, of about 500 mg to about 5000 mg.

20. The method according to claim 15, wherein the oral formulation comprises sustained-release or extended-release metformin hydrochloride.

21. The method according to claim 15, wherein the insulin sensitizer is a glitazone.

22. The method according to claim 21, wherein said glitazone is pioglitazone.

23. The method according to claim 21, wherein said glitazone is rosiglitazone.

24. The method according to claim 15, wherein said metformin or pharmaceutically active salt or ester thereof is metformin HCl.

25. A method for treating diabetes and related disorders in a mammal, comprising administering to said mammal an oral formulation comprising pioglitazone, glimepiride, and metformin HCl.

26. The method of claim 25, wherein said pioglitazone is present in said oral formulation in an amount of about 2 mg to about 120 mg.

27. The method of claim 26, wherein said pioglitazone is present in said oral formulation in an amount of about 15 mg.

28. The method of claim 25, wherein said glimepiride is present in said oral formulation in an amount of about 0.5 mg to about 40 mg.

29. The method of claim 28, wherein said glimepiride is present in said oral formulation in an amount of about 2 mg.

30. The method of claim 25, wherein said metformin HCl is present in said oral formulation in an amount of about 500 mg to about 5000 mg.

31. The method of claim 25, wherein said metformin HCl is present in said oral formulation in an amount of about 500 mg to about 850 mg.

32. The method of claim 25, wherein said metformin HCl is present in said oral formulation in an amount of about 500 mg.

33. The method of claim 25, wherein said metformin HCl is present in said oral formulation in an amount of about 850 mg.

34. The method of claim 25, wherein the oral formulation comprises sustained-release or extended-release metformin hydrochloride.

35. The method of claim 25, wherein said mammal is a human.

36. A method for treating diabetes and related disorders in a mammal, comprising administering to said mammal the pharmaceutical formulation of claim 1.

37. The method of claim 36, wherein said mammal is a human.
